(19) United States
(12) Patent Application Publication
(10) Pub. No.: US 2021/0225474 A1
(43) Pub. Date: Jul. 22, 2021

(54) ELECTRONIC PRESCRIPTION VOUCHER SYSTEM AND METHOD OF GENERATING ELECTRONIC PRESCRIPTION VOUCHER

(71) Applicant: TROY GROUP, INC., Costa Mesa, CA (US)

(72) Inventors: Craig Allan Baldassare, McMurray, PA (US); Jamison Burgess A Benson, Natrona Heights, PA (US); Robert David Kaleugher, Pittsburgh, PA (US)

(21) Appl. No.: 16/923,390

(22) Filed: Jul. 8, 2020

Related U.S. Application Data
(60) Provisional application No. 62/962,082, filed on Jan. 16, 2020.

(51) Int. Cl. G16H 20/10; G16H 10/60; H04W 4/14; H04L 12/58; G06K 7/14

(52) U.S. Cl. CPC G16H 20/10; G16H 10/60; G06K 7/1417; H04L 51/046; H04L 51/38; H04W 4/14

(57) ABSTRACT

An electronic prescription voucher system provides the security of an electronic prescription while giving a patient control over the selection of a pharmacy and when to fill the prescription after the patient has left a healthcare provider's office. A cloud-based voucher service provides the patient with a printable paper-based prescription voucher or an electronic prescription voucher before he leaves the provider's office. The patient will be able to fill the prescription at a time of his choosing at an EHR or EMR participating pharmacy by using his smartphone to scan a QR barcode on the paper prescription voucher or enter a prescription ID from the prescription voucher on his smartphone. The prescription is then sent to the pharmacy chosen by the patient. The patient will be notified through the system when the prescription is ready to be picked up.

### CROSS REFERENCE TO RELATED APPLICATIONS

Applicant claims the benefit of provisional application U.S. Ser. No. 62/962,082 filed Jan. 16, 2020 for issuing and filing an electronic prescription through a patient prescription voucher system. The entire application is incorporated herein by reference.

### BACKGROUND OF THE INVENTION

### 1. Field of the Invention

The present invention relates generally to a prescription voucher system that utilizes the E-prescribing facilities of an electronic health record system (EHR) or an electronic medical record system (EMR) to submit a healthcare provider-issued paper prescription to a pharmacy participating in the EHR or EMR service.

### 2. Description of Related Art

Electronic health record (EHR) systems in use today are designed to store patient and population health information in digital format in a systemized collection. These records are then shared across different heath care settings. The records are shared through network-connected, enterprise-wide information systems or other information networks and exchanges. EHRs may include a range of data including demographics, medical history, medication and allergies, immunization status, laboratory test results, radiology images, vital signs, personal statistics like age and weight, and billing information.

EHR systems are designed to store data accurately and to capture the state of a patient across time. The system eliminates the need to track down a patient's previous paper medical records and assists in assuring data is accurate and legible. It can reduce risk of data replication as there is only one modifiable file. This means the file is more likely up-to-date, and decreases risk of lost paperwork. Due to the digital information being searchable and in a single file, EMRs are more effective when extracting medical data for the examination of possible trends and long-term changes in a patient. Population-based studies of medical records may also be facilitated by wide-spread adoption of EHRs.

A similar system to EHRs are electronic medical records. Electronic medical record (EMR) systems are the digital equivalent of paper records or charts at a clinician's office. EMRs typically contain general information such as treatment and medical history about a patient as it is collected by the individual medical practice.

An electronic prescribing function is usually associated with either the EMR or EHR technology. The electronic prescribing function is a computer-based electronic generation, transmission and filing of a medical prescription which takes the place of paper and faxed prescriptions. Electronic prescribing (e-prescribing or e-Rx) allows a physician, pharmacist, nurse practitioner or physician assistant to use digital prescription software to electronically transmit a new prescription or renewal authorization to a community or mail order pharmacy. The system provides the ability to send error-free, accurate and understandable prescriptions electronically from the healthcare provider to the pharmacy. E-prescribing is intended to reduce the risks associated with traditional prescription script writing. It is also one of the major reasons for a push for electronic medical records. By sharing medical prescription information, e-prescribing seeks to connect a patient's team of healthcare providers to facilitate knowledgeable decision making.

EMRs work well in practice. However, they are limited because they do not easily travel outside that practice. In fact, a patient's medical record might even have to be printed out and mailed to another provider.

EHRs provide not only data that it contains, but it is capable of sharing that data electronically. EHRs make health information instantly accessible to authorize providers across practices and health organizations, helping to inform clinical decisions and coordinate care. An EHR can be shared with all clinicians and organizations involved in a patient's care, such as labs, specialists, imaging facilities, pharmacies, emergency facilities and school and workplace clinics.

EHR systems are the popular trend in the medical industry with healthcare providers because these systems provide critical data that informs clinical decisions and the system helps coordinate care between all providers in the healthcare ecosystem for a certain patient. The e-prescribing system that forms a part of this larger EHR system is also intended to connect a patient's team of healthcare providers to facilitate knowledgeable decision making. The basic components of the e-prescribing system are a prescriber, typically a physician, which enters data into the system at a provider terminal 11, a transaction hub 13, designed to maintain a master patient index and functions to route inquiries to a matched pharmacy benefit manager. The transaction hub also maintains an index of enrolled pharmacies and routes prescriptions to desired pharmacies 17, which have implemented electronic prescribing software, and to a pharmacy benefit manager 15.

A healthcare provider 11 provides patient demographics and prescription information to the transaction hub. The transaction hub, in turn, provides eligibility, formulary, medication history and fill status to the healthcare provider. The transaction hub 13 provides prescription data 27 to the pharmacy 17. The pharmacy, in turn, provides prescription fill status 29 to the transaction hub 13. The transaction hub further provides patient information inquiry 35 to the pharmacy benefit manager 15 which responds with eligibility, formulary and medication history 33 to the transaction hub.

The transaction hub 13 is the common link between all the other elements—the healthcare provider terminal, pharmacy benefit manager and the pharmacy. After the healthcare provider uploads new prescription information to the patient file in the transaction hub, the transaction hub 13 will send information about the prescription to the pharmacy benefit manager 15 who will respond with information on the patient eligibility, formulary and medication history. The transaction hub then sends this information to the healthcare provider authorizing the prescription as well as sending the prescription information to the patient's primary pharmacy.

What is missing in this electronic prescription system is a patient's interaction and control of his prescription after the healthcare provider 11 enters the prescription in the electronic prescribing (e-Rx) system. The patient has no control of the process after that data is entered into the system.

The present invention provides a patient the ability to fill his prescription after he leaves the healthcare provider's facilities by sending the prescription to the pharmacy at a time of his choosing by a web portal or by a mobile application.

### SUMMARY OF THE INVENTION

An electronic prescription system and method that leaves the choice of pharmacy and time of filling in the hands of the patient, like a paper prescription, while providing the speed and security of filling a prescription, as in an electronic prescription system. A voucher service is in communication with an EMR system to receive prescriptions entered into the EMR prescription system by a healthcare provider. The voucher service produces either a printable prescription voucher or an electronic prescription voucher before the patient leaves the provider's location, based on the patient's choice. The patient then has the option of filling the prescription at an EMR prescription pharmacy of his choice at a time of his choosing. If the patient has a paper copy of the prescription voucher, he can scan a QR barcode on the paper voucher into the voucher system using a smartphone. If he has an electronic prescription voucher, he can enter the prescription ID number into his smartphone. The system returns a list of participating pharmacies in his area. After the patient chooses a pharmacy, the prescription is electronically routed that pharmacy. The chosen pharmacy fills the prescription and notifies the patient through the prescription voucher system and the healthcare provider EMR prescription system that the prescription is ready to be picked up.

### BRIEF DESCRIPTION OF THE DRAWINGS

The exact nature of this invention, as well as its advantages, will become better understood by reference to the following detailed description when considered in conjunction with the accompanying drawings, in which like referenced numerals designate like parts throughout the figures thereof, and wherein:

FIG. 1 is a block diagram illustration of the prescription voucher system of the present invention connected for interaction with an electronic prescribing system.

FIG. 2 is a block diagram of the basic elements of the present invention.

FIGS. 3A and 3B is a flow chart illustrating the basic functions of the present invention.

FIG. 4 is a mock up of a proposed paper electronic prescription voucher.

FIG. 5 is a proposed screen mock up of an electronic prescription voucher received by email or smartphone.

FIG. 6 is a proposed screen mock up of an electronic prescription voucher requiring response from the patient to fill a prescription.

FIG. 7 is a proposed screen mock up of an email on a smartphone for selecting a pharmacy.

FIG. 8 is a proposed screen mock up of an email on a smartphone after the prescription has been sent to a selected pharmacy.

### DETAILED DESCRIPTION OF THE PREFERRED EMBODIMENTS

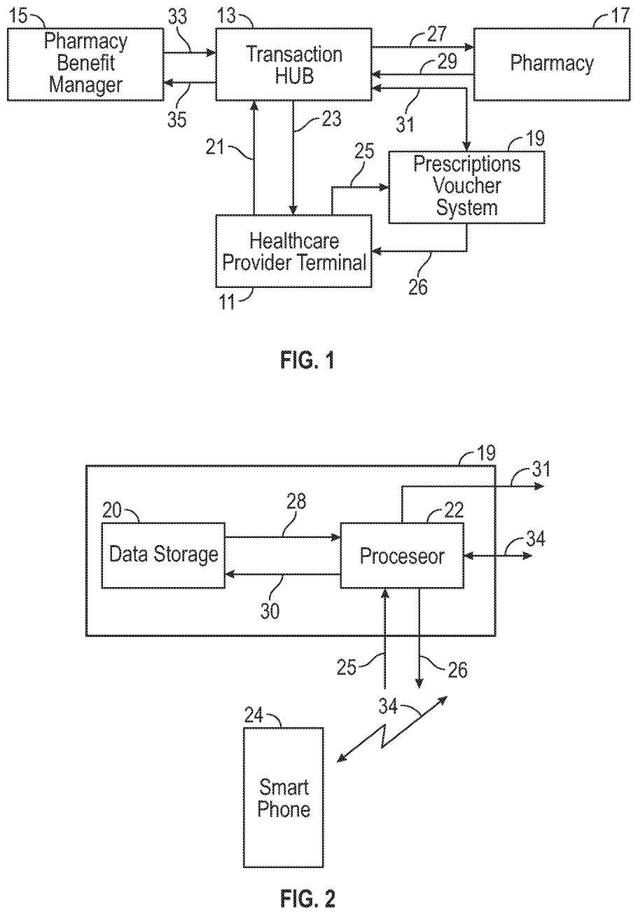

FIG. 1 illustrates the electronic interconnection of the prescription voucher system 19 of the present invention with the elements of a typical electronic prescribing system (e-Rx), which is a computer-based electronic generation, transmission and filing of a medical prescription that takes the place of paper and faxed prescriptions. The electronic prescribing system typically contains the elements of a healthcare provider terminal 11 communicating with a transaction hub 13 of an e-prescribe network, such as SureScripts®, for example, which communicates with an authorized pharmacy 17 and a pharmacy benefit manager 15. Once a prescription is entered into the system by the healthcare provider terminal 11, the patient interaction occurs only at the pharmacy when the patient picks up the filled prescription.

The prescription voucher system 19 according to the present invention gives the patient control of the prescribing process. At the time a prescription is entered into the electronic prescribing system at the healthcare provider 11, the patient is given the option to receive a prescription voucher. If the patient chooses to receive a prescription voucher, they will receive either a paper-based or electronic prescription voucher which will allow them to electronically send a prescription to a pharmacy of their choice and on their own timeline.

Upon the patient choosing a prescription voucher option, the healthcare provider 11 enters the prescription as a voucher Rx. The healthcare provider terminal 11 includes an application programming interface, which is a communication protocol between the electronic prescription software and the prescription voucher system software 19 to essentially simplify the implementation of communication between the two systems.

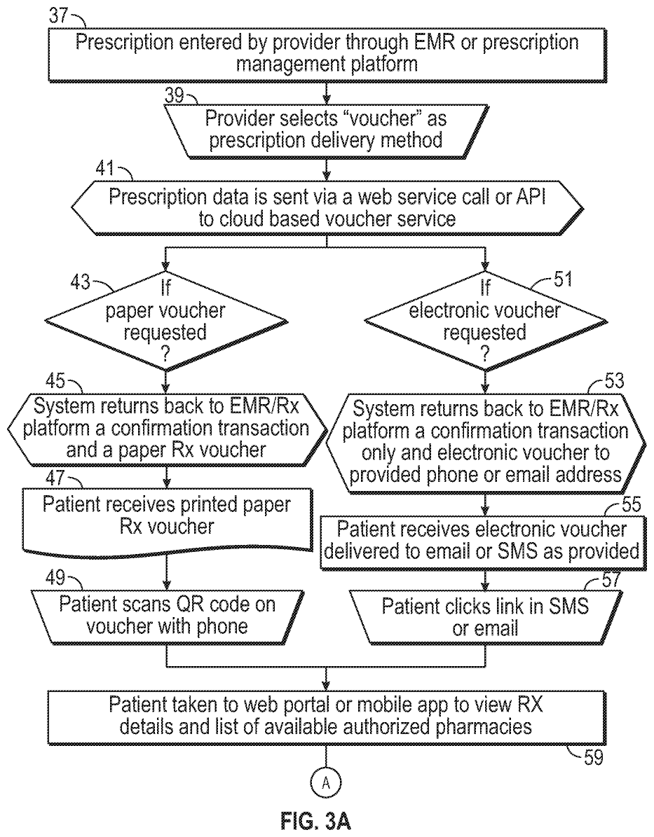

This facilitate the prescription data entered at the healthcare provider terminal 11 to be sent over a communication link 25 to the prescription vouchers system 19, which may be cloud-based, to be stored there awaiting additional instructions from the patient. Upon the prescription being sent to the prescription voucher system 19 and stored in the cloud database, a printable paper-based version of the prescription voucher is sent over wireless communication link 26 to the healthcare provider terminal 11. If the patient selected a text or email copy of the prescription voucher, that is returned to the patient's email address or smartphone 24 (FIG. 2).

FIG. 2 illustrates the components of the prescription voucher system 19 as containing data storage 20 and a processor 22 which connects with the electronic prescribing system of FIG. 1. The processor 22 receives the prescription data over communication link 25 from a healthcare provider terminal 11 and stores that information in the data storage 20. Processor 22 sends a printable paper-based version of the prescription voucher as a text or email over link 26 back to the healthcare provider and to the patient's smartphone 24 over link 34.

The patient then has a copy of the prescription voucher, either in paper form or in a text or email on his smartphone 34 or on his PC before he leaves the health providers office. When the patient decides to fill the prescription, he can access the portal or mobile application on his smartphone to communicate with the prescription voucher system 19.

The prescription voucher system 19 communicates with the transaction hub 13 of the e-RX over communication link 31. The e-Rx then routes the prescription to the pharmacy chosen by the patient.

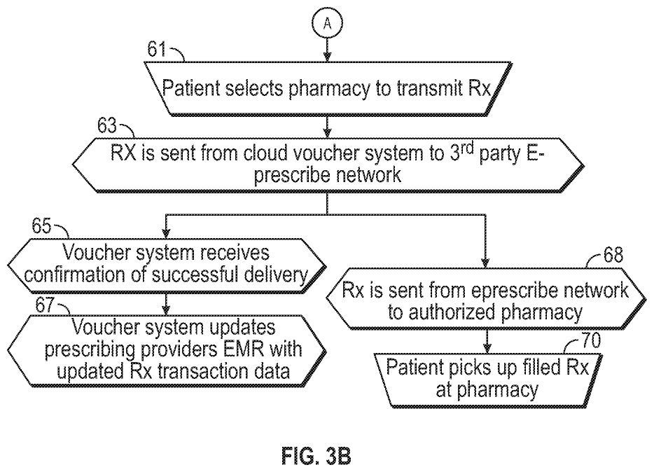

Referring now to FIG. 3, a detailed information flow of the system described in FIGS. 1 and 2 is illustrated. The prescription voucher system 19 of the present invention is activated only upon the healthcare provider selecting “voucher” 39 at the time the provider enters a prescription into the electronic prescribing system 37. Upon selecting “voucher”, the prescription data entered by the provider is sent by way of a web service call or an application programming interface communication link, to the prescription voucher system 19, which may be cloud-based 41. That data is entered into the data storage 20 of the prescription voucher system 19.

The patient, while in the healthcare provider's office, after having selected the prescription voucher system, will select either receiving a paper voucher 43 or an electronic voucher 51. Upon the selecting a paper voucher, the prescription voucher system 19, a confirmation transaction and an electronic version of a paper prescription voucher 45, to the healthcare provider terminal 11, that then prints out the prescription voucher 47.

If the patient selected an electronic voucher, the prescription voucher system 19 returns a confirmation transaction to the a mobile phone 24 or to an email address. The electronic voucher system 19 provides a prescription voucher 55 to the patient's phone 24 by text or email.

If the patient has a printed paper prescription voucher and intends to fill that prescription, the patient will scan the QR code on the paper voucher with his smartphone 49. If the patient has the electronic voucher on his smartphone or as an email on his PC, the patient will click the link in the electronic voucher or email to fill his prescription 57.

Either way, the patient is taken to a dedicated web portal or a mobile app to view his prescription details and a list of available authorized pharmacies 59 as well as other helpful information, such as pricing, for example. The patient then must select the pharmacy he wants to fill his prescription 61. Upon selection of a pharmacy, the prescription as stored in the database 25 of the prescription voucher system 19, is sent to the transaction hub 13 over bilateral wireless communication link 31. The e-prescribing network then sends a prescription to the selected authorized pharmacy 69 and a confirmation of successful delivery to the prescription voucher system 19. Voucher system 19 then updates the records of the transaction hub with prescription transaction data 67 after the patient picks up the filled prescription at the selected pharmacy 70.

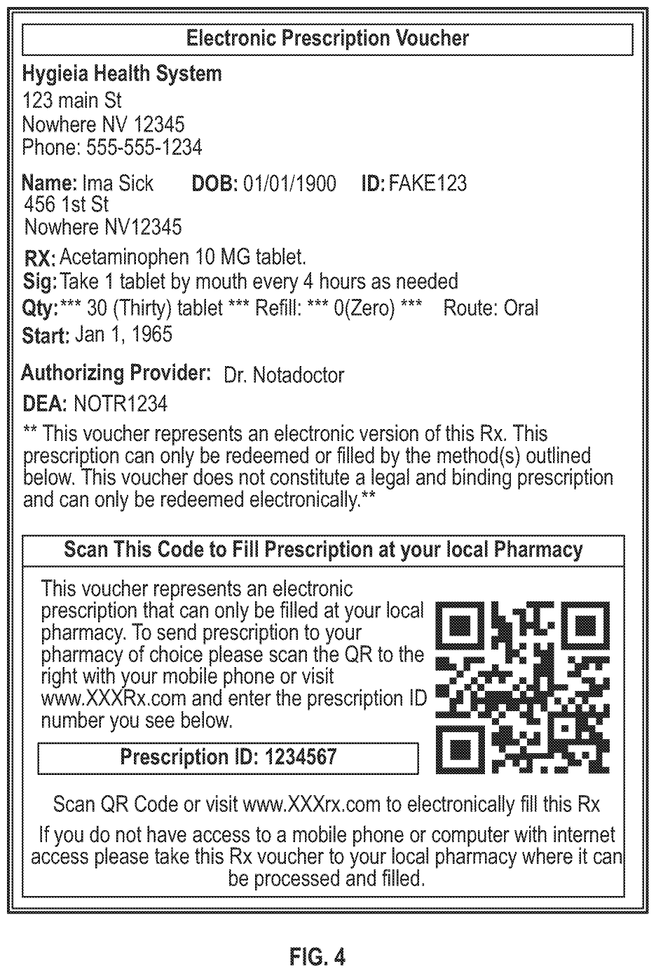

FIG. 4 illustrates the type of information contained on a paper prescription voucher after being printed at the healthcare provider terminal 11. It contains a barcode which the patient can scan with his smartphone 24 sending the prescription to the prescription voucher system 19. An alternative method of sending the prescription to the prescription voucher system 19 is to key in the prescription ID listed on the paper voucher, as illustrated in FIG. 4.

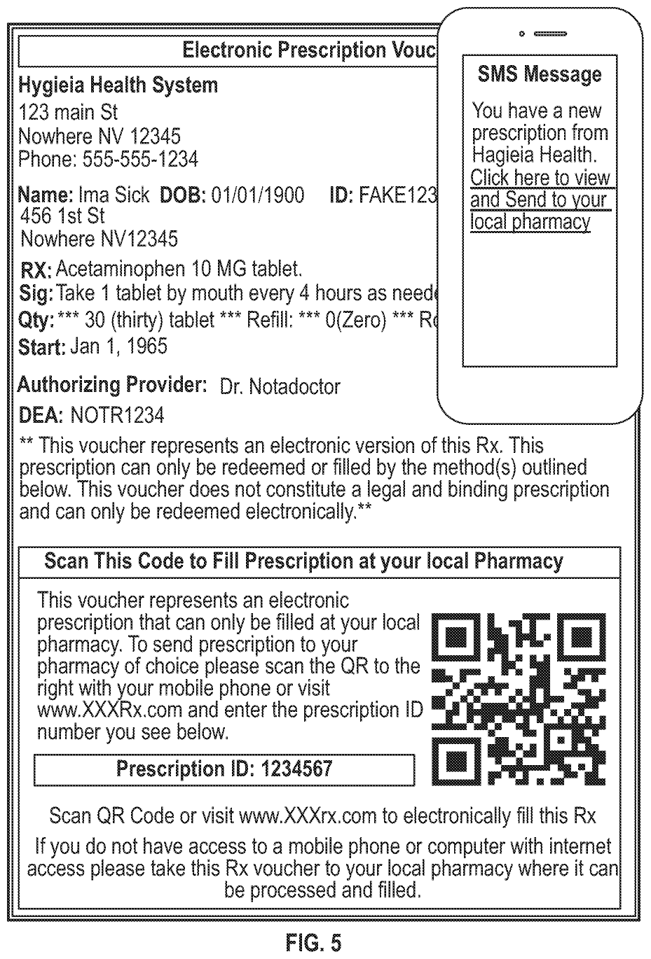

FIG. 5 illustrates the electronic version of the prescription voucher, which may be an email or text based, that provides the patient with a click location to fill the prescription.

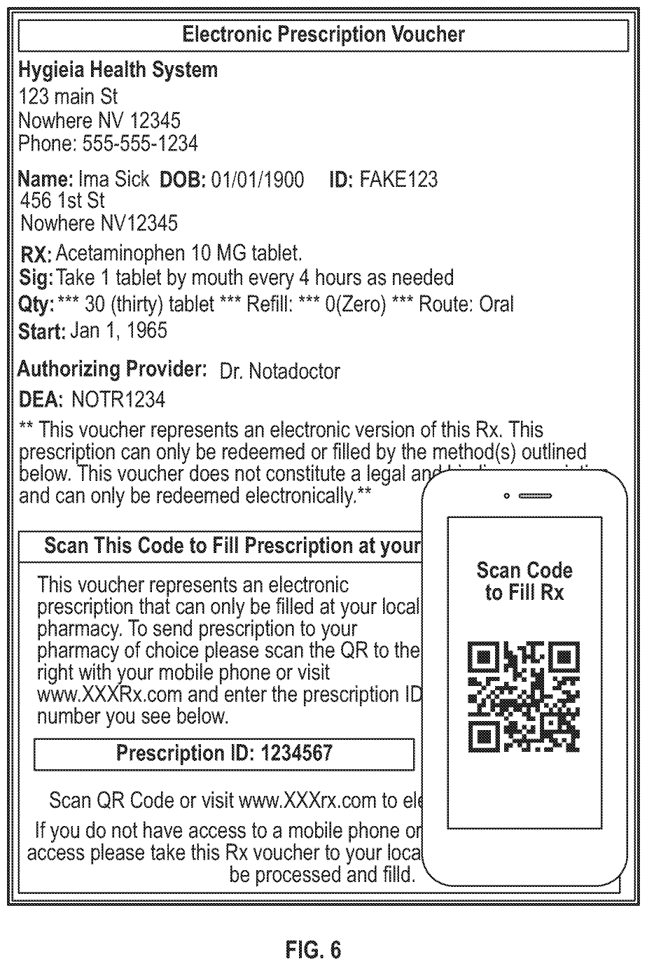

FIG. 6 illustrates a paper prescription voucher which prompts scanning of the code on the prescription voucher to fill a prescription, sending it electronically into the electronic prescribing system. In the alternative, the patient can take this paper electronic prescription voucher to his pharmacy of choice, which pharmacy will then scan the prescription into their system and fill the prescription on-site. This alternative would be appropriate only if the patient does not have access to the Internet.

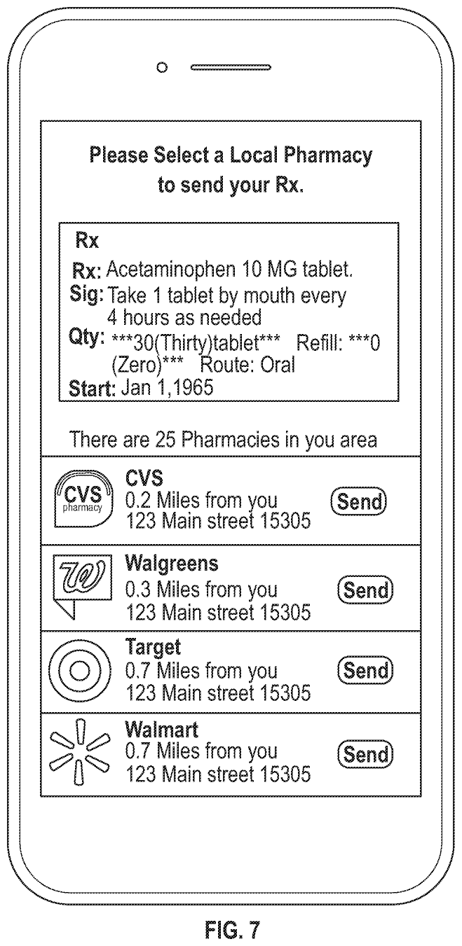

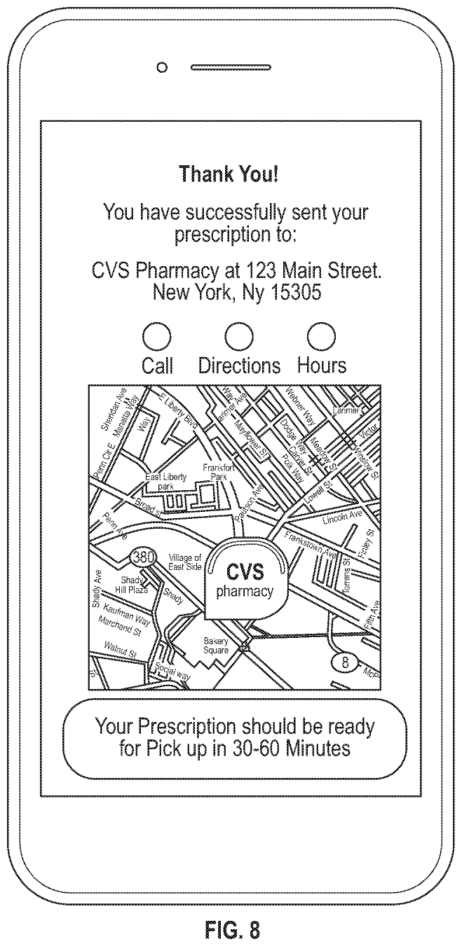

FIG. 7 illustrates a proposed screen that comes up after the patient enters his prescription, either by scanning or by clicking, as illustrated in FIGS. 5 and 6. FIG. 7 requires the patient to select the authorized pharmacy he wants to fill his prescription. Upon selecting the pharmacy, the prescription voucher system 19 communicates this information to the transaction hub 13, which then authorizes the selected pharmacy 17 over communication link 27. Upon that information being submitted to the selected pharmacy, the patient will receive a screen as shown in FIG. 8, which shows the name of the pharmacy, its location and the time that the prescription can be picked up, for example.

What is claimed is:

1. A prescription voucher system connected to an electronic medical record system subscribed to by a healthcare provider, the prescription voucher system comprising:
a data storage device; and
a processor connected to the data storage device for bilateral communication, the processor also connected for bilateral communication with the healthcare provider, the processor further connected for communication with a plurality of pharmacies, and the processor connected to the electronic medical record system.

2. The prescription voucher system of claim 1 wherein the data storage device stores patient and prescription data entered into the electronic medical record system by the healthcare provider.

3. The prescription voucher system of claim 2 further comprising a smartphone containing a prescription voucher user application for wirelessly bilateral communication with the processor.

4. The prescription voucher system of claim 1 wherein the data storage device stores a prescription voucher format capable of being populated by patient and prescription data entered into the electronic medical record system by the healthcare provider.

5. A prescription voucher system connected for bilateral communication with an electronic medical record system having a healthcare provider terminal bilaterally communicating with a transaction hub in the electronic medical record system, the transaction hub bilaterally communicating with a pharmacy benefit manager and bilaterally communicating with a plurality of authorized pharmacies, the prescription voucher system comprising:
a data storage device; and
a processor connected for bilateral communication with the data storage device, the processor also connected for bilateral communication with the healthcare provider terminal, the processor further connected for communication with the transaction hub.

6. The prescription voucher system of claim 5 wherein the data storage device stores patient and prescription data entered into the transaction hub and the prescription voucher system by the healthcare provider terminal.

7. The prescription voucher system of claim 6 further comprising a smartphone contacting a prescription voucher user application for wireless bilateral communication.

8. The prescription voucher system of claim 5 wherein the data storage device stores a prescription voucher format capable of being populated by patient and prescription data generated by the healthcare provider terminal.

9. A method of providing an electronic prescription voucher to a patient for filling a prescription from a healthcare provider, the steps of the method comprising:
the healthcare provider selecting voucher as an option for a prescription delivery method;
prescription data sent to a voucher invoice system as a result of that selection;
patient selects either a printable prescription voucher or an electronic prescription voucher to be returned;
if a paper prescription voucher is selected, the voucher system sends a confirmation and a printable paper prescription voucher to the electronic medical system; and
patient receives a printed paper prescription voucher from the healthcare provider.

10. The method of claim 9 further comprising:
if an electronic prescription voucher is selected, the voucher system sends a confirmation to the electronic medical system and an electronic voucher to the patient's phone by short message service (text message) or to the patient's email address; and
patent receives the electronic prescription voucher at his email address or by text message.

11. The method of claim 9, further comprising:
patient scans a QR code on the paper prescription voucher with his smartphone.

12. The method of claim 10 further comprising:
patient clicks link on the electronic prescription voucher received by email or smartphone.

13. The method of claim 11, further comprising:
QR code scan causes return to smartphone of prescription details and authorized pharmacies;
patient selects on smartphone a pharmacy to fill the prescription;
the prescription details in the voucher system are then sent to the electronic medical record system;
the voucher system receives confirmation that the prescription details were received by the electronic medical records system;
the electronic medical records system sends the prescription to the selected authorized pharmacy;
patient picks up filled prescription from the pharmacy; and
the voucher system updates the electronic medical records system with prescription transaction data.

14. The method of claim 12, further comprising:
patient clicks on link, causes return of prescription details and authorized pharmacies;
patient selects pharmacy to fill prescription;
the prescription details in the voucher system are then sent to the electronic medical record system;
the voucher system receives confirmation that the prescription details were received by the electronic medical records system;
the electronic medical records system sends the prescription to the selected authorized pharmacy;
patient picks up filled prescription from the pharmacy; and
the voucher system updates the electronic medical records system with prescription transaction data.
